(19) United States
(12) Patent Application Publication
(10) Pub. No.: US 2011/0184466 A1
(43) Pub. Date: Jul. 28, 2011

(54) Spinal Instrumentation Systems and Methods of Making and Using the Same

(76) Inventor: Harry C. Weiser, Albany, GA (US)

(21) Appl. No.: 13/013,856

(22) Filed: Jan. 26, 2011

Related U.S. Application Data
(60) Provisional application No. 61/336,729, filed on Jan. 26, 2010.

(51) Int. Cl. A61B 17/70; A61B 17/88

(52) U.S. Cl. 606/264; 606/246; 606/279

(57) ABSTRACT

Spinal instrumentation systems are disclosed. Methods of making and using spinal instrumentation systems are also disclosed.

### CROSS-REFERENCE TO RELATED APPLICATIONS

This patent application claims the benefit of priority to U.S. Provisional Patent Application Ser. No. 61/336,729 filed on Jan. 26, 2010 and entitled “EXPANDABLE ROD DESIGN AND CONCEPT FOR SPINAL INSTRUMENTATION,” the subject matter of which is hereby incorporated by reference in its entirety.

### TECHNICAL FIELD

The present invention relates to spinal instrumentation systems, as well as methods of making and using spinal instrumentation systems.

### BACKGROUND

Kanada systems for use in corpectomy procedures are known. The Kanada system is a lateral spinal instrumentation system that is used for corpectomies of either the thoracic or lumbar vertebral bodies as a result of tumor, trauma, etc. The Kanada system uses rods that affix to screws previously driven into the lateral body of the vertebral body. An exemplary set of vertebral bodies and an exemplary Kanada system are shown in FIGS. 1-2.

As shown in FIG. 1, exemplary adjacent vertebral bodies 11 and 12 are separated by a site of a corpectomy 10 (hereinafter, “corpectomy site 10”) therebetween. Although exemplary adjacent vertebral bodies 11 and 12 and corpectomy site 10 are shown as having square shapes, it should be understood that a given vertebral body 11 and/or 12 and a given corpectomy site 10 may have any shape.

As shown in FIG. 2, exemplary Kanada system 80 comprises two separate titanium rods 13 and 14, four rod stabilizing members 81 (e.g., nuts 81) and four screws 16 attaching opposite ends of titanium rods 13 and 14 to exemplary adjacent vertebral bodies 11 and 12 via rod stabilizing members 81. An implant 15 is shown positioned within corpectomy site 10 between exemplary adjacent vertebral bodies 11 and 12.

Use of the Kanada system, such as exemplary Kanada system 80, involves sizing the rods (i.e., titanium rods 13 and 14), cutting the rods (i.e., titanium rods 13 and 14), and then affixing the rods (i.e., titanium rods 13 and 14) to exemplary adjacent vertebral bodies 11 and 12 via screws 16 and rod stabilizing members 81 (e.g., nuts 81). The process of connecting rods (i.e., titanium rods 13 and 14) to exemplary adjacent vertebral bodies 11 and 12 via screws 16 and rod stabilizing members 81 (e.g., nuts 81) involves up to four separate tightening and torquing steps for each of the four rod stabilizing members 81 (e.g., nuts 81). The process is very cumbersome and burdensome.

What is needed in the art is a system that simplifies the process of connecting adjacent vertebral bodies (e.g., exemplary adjacent vertebral bodies 11 and 12) to one another and eliminates one or more steps needed in the Kanada system.

### SUMMARY

The present invention addresses the need for a system that simplifies the process of connecting portions of one or more vertebral bodies (e.g., exemplary adjacent vertebral bodies 11 and 12) to one another. The systems and methods of the present invention enable the efficient connection of portions of one or more vertebral bodies (e.g., exemplary adjacent vertebral bodies 11 and 12) to one another and elimination of one or more steps required in the known Kanada system.

The device of the present invention eliminates the entire process of fitting rods (i.e., titanium rods 13 and 14), measuring rods (i.e., titanium rods 13 and 14), and cutting rods (i.e., titanium rods 13 and 14) in the operating room. The device of the present invention also eliminates the need to distract or compress (i.e., adjust a position thereof) of up to four rod positions once in place.

The device of the present invention is connected to portions of one or more vertebral bodies (e.g., exemplary adjacent vertebral bodies 11 and 12) via screws, tightened in place, and then distracted or compressed (i.e., adjust a position thereof) via a counterscrew wormgear. The counterscrew concept extends and retracts opposite arms/footplates of the device without temporarily disconnecting a given arm/footplate from a given vertebral body portion. The device of the present invention is simply placed over the desired vertebral body portions, secured into place with the screws, tightened/torqued with nuts and then either extended for distraction or shortened for compression. The device of the present invention eliminates the steps of measuring the rods, cutting them and then having to individually distract/compress. It reduces the whole rod placement to about one step.

Accordingly, the present invention is directed to devices for efficiently and effectively connecting portions of one or more vertebral bodies to one another. In one exemplary embodiment, the device for connecting portions of one or more vertebral bodies to one another comprises a first member operatively adapted and sized to connect to a first portion of a vertebral body; a second member operatively adapted and sized to connect to a second portion of the vertebral body; and a counterscrew wormgear comprising (i) a nut positioned along a central portion of the counterscrew wormgear, (ii) a set of right-handed threads extending along a first length of the counterscrew wormgear between the nut and a first end of the counterscrew wormgear, and (iii) a set of left-handed threads extending along a second length of the counterscrew wormgear between the nut and a second end of the counterscrew wormgear opposite the first end of said counterscrew wormgear; the counterscrew wormgear being engaged with the first and second members so as to cause the first and second members to move toward or away from one another depending on a direction of rotation of the counterscrew wormgear via the nut.

In a further exemplary embodiment, the device for connecting portions of one or more vertebral bodies to one another comprises a first engaging member comprising (i) a first engaging member body, (ii) a first set of fastening members positioned proximate a first end of the first engaging member and thru the first engaging member body, (iii) one or more first arm members extending from the first engaging member body and toward a second end of the first engaging member, and (iv) at least one first set of teeth positioned along a surface of the one or more first arm members; a second engaging member comprising (i) a second engaging member body, (ii) a second set of fastening members positioned proximate a first end of the second engaging member and thru the second engaging member body, (iii) one or more second arm members extending from the second engaging member body and toward a second end of the second engaging member, and (iv) at least one second set of teeth positioned along a surface of the one or more second arm members; a counterscrew wormgear comprising (i) a nut positioned along a central portion of the counterscrew wormgear, (ii) a set of right-handed threads extending along a first length of the counterscrew wormgear between the nut and a first end of the counterscrew wormgear, and (iii) a set of left-handed threads extending along a second length of the counterscrew wormgear between the nut and a second end of the counterscrew wormgear opposite the first end of the counterscrew wormgear, the counterscrew wormgear being engaged with the first and second engaging members so as to cause the first and second engaging members to move toward or away from one another depending on a direction of rotation of the counterscrew wormgear via the nut; and a tubular housing member comprising a first housing end, a second housing end opposite the first housing end, one or more housing walls extending between the first and second housing ends, a channel extending through the tubular housing member from the first housing end to the second housing end, and at least one opening therein so as to allow access to the nut; wherein (i) a portion of the first engaging member, (ii) a portion of the second engaging member, and (iii) the counterscrew wormgear are positioned within the channel.

The present invention is further directed to kits for connecting portions of one or more vertebral bodies to one another. In one exemplary embodiment, the kit for connecting portions of one or more vertebral bodies to one another, comprises (I) a device comprising a first member operatively adapted and sized to connect to a first portion of one or more vertebral bodies; a second member operatively adapted and sized to connect to a second portion of the one or more vertebral bodies; and a counterscrew wormgear comprising (i) a nut positioned along a central portion of the counterscrew wormgear, (ii) a set of right-handed threads extending along a first length of the counterscrew wormgear between the nut and a first end of the counterscrew wormgear, and (iii) a set of left-handed threads extending along a second length of the counterscrew wormgear between the nut and a second end of the counterscrew wormgear opposite the first end of the counterscrew wormgear; the counterscrew wormgear being engageable with the first and second members so as to cause the first and second members to move toward or away from one another depending on a direction of rotation of the counterscrew wormgear via the nut; and (II) a tool for engaging with and turning the nut of the counterscrew wormgear. The kit may further comprise a number of additional kit components including, but not limited to, multiple fasteners operatively adapted and sized to connect a first and second members or engaging members to first and second portion of one or more vertebral bodies (e.g., multiple screws); and one or more implants for positioning between first and second portions of one or more vertebral bodies.

The present invention is further directed to methods of making the disclosed devices and kits for connecting portions of one or more vertebral bodies to one another. In one exemplary embodiment, the method of making a device for connecting portions of one or more vertebral bodies to one another comprises combining (1) a first member operatively adapted and sized to connect to a first portion of a vertebral body, (2) a second member operatively adapted and sized to connect to a second portion of the vertebral body, and (3) a counterscrew wormgear comprising (i) a nut positioned along a central portion of the counterscrew wormgear, (ii) a set of right-handed threads extending along a first length of the counterscrew wormgear between the nut and a first end of the counterscrew wormgear, and (iii) a set of left-handed threads extending along a second length of the counterscrew wormgear between the nut and a second end of the counterscrew wormgear opposite the first end of said counterscrew wormgear; the counterscrew wormgear being engageable with the first and second members so as to cause the first and second members to move toward or away from one another depending on a direction of rotation of the counterscrew wormgear via the nut. The method of making a device for connecting portions of one or more vertebral bodies to one another may further comprise one or more additional steps. Suitable steps include, but are not limited to, engaging the counterscrew wormgear with the first and second members; positioning the engaged counterscrew wormgear and first and second members within a housing member; and providing multiple fasteners (e.g., screws) operatively adapted and sized to connect the first and second members to first and second portion of the one or more vertebral bodies.

The present invention is even further directed to methods of using the disclosed devices and kits for connecting portions of one or more vertebral bodies to one another. In one exemplary embodiment, the method of using a device for connecting portions of one or more vertebral bodies to one another comprises a method of adjusting a position of a first portion of one or more vertebral bodies relative to a second portion of the one or more vertebral bodies. The method may comprise (I) attaching first and second members of a device to first and second portions of the one or more vertebral bodies respectively, wherein the device comprises a first member operatively adapted and sized to connect to a first portion of a vertebral body; a second member operatively adapted and sized to connect to a second portion of the vertebral body; and a counterscrew wormgear comprising (i) a nut positioned along a central portion of the counterscrew wormgear, (ii) a set of right-handed threads extending along a first length of the counterscrew wormgear between the nut and a first end of the counterscrew wormgear, and (iii) a set of left-handed threads extending along a second length of the counterscrew wormgear between the nut and a second end of the counterscrew wormgear opposite the first end of said counterscrew wormgear; the counterscrew wormgear being engaged with the first and second members so as to cause the first and second members to move toward or away from one another depending on a direction of rotation of the counterscrew wormgear via the nut; and (II) turning the nut of the counterscrew wormgear.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a frontal view of exemplary portions of one or more vertebral bodies and a site of a corpectomy therebetween;

FIG. 2 depicts a frontal view of components of the Kanada system of the prior art;

FIG. 3 depicts a frontal view of an exemplary expandable rod device of the present invention;

FIG. 4 depicts a side view of the exemplary expandable rod device shown in FIG. 3 as viewed along direction A;

FIGS. 5A-5C depict frontal, side and ends views of an exemplary housing component suitable for use in the exemplary expandable rod device shown in FIG. 3;

FIG. 6 depicts exemplary footplate components (also referred to herein as first and second members or engaging members) of the exemplary expandable rod device shown in FIG. 3 as viewed with an upper surface (and upper wall) of the exemplary housing component removed;

FIG. 7 depicts an exemplary wormgear and exemplary nut suitable for use in the exemplary expandable rod device shown in FIG. 3;

FIG. 8 depicts the exemplary wormgear and exemplary nut shown in FIG. 7 positioned relative to one exemplary footplate component (or first member or first engaging member) of the exemplary expandable rod device shown in FIG. 3; and

FIG. 9 depicts an exemplary tool suitable for engaging with and causing movement of the exemplary wormgear and exemplary nut shown in FIGS. 3 and 7.

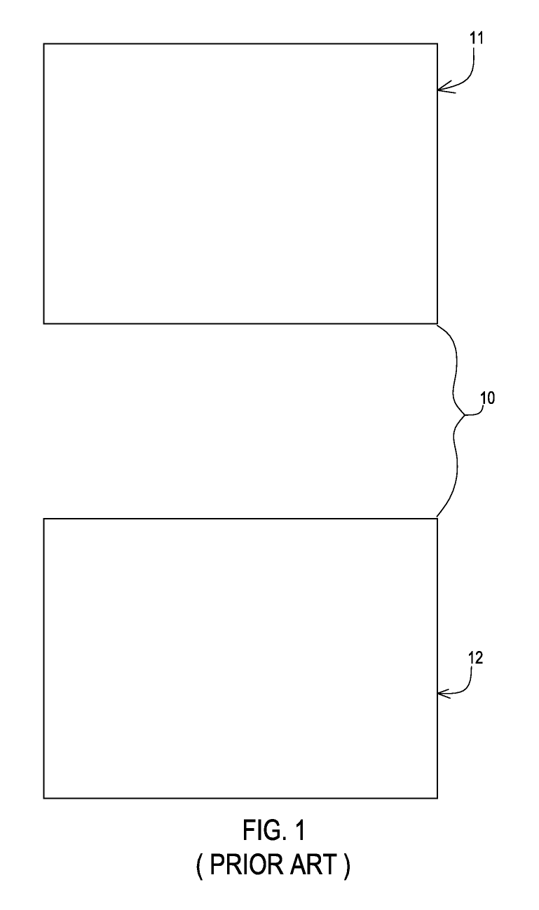

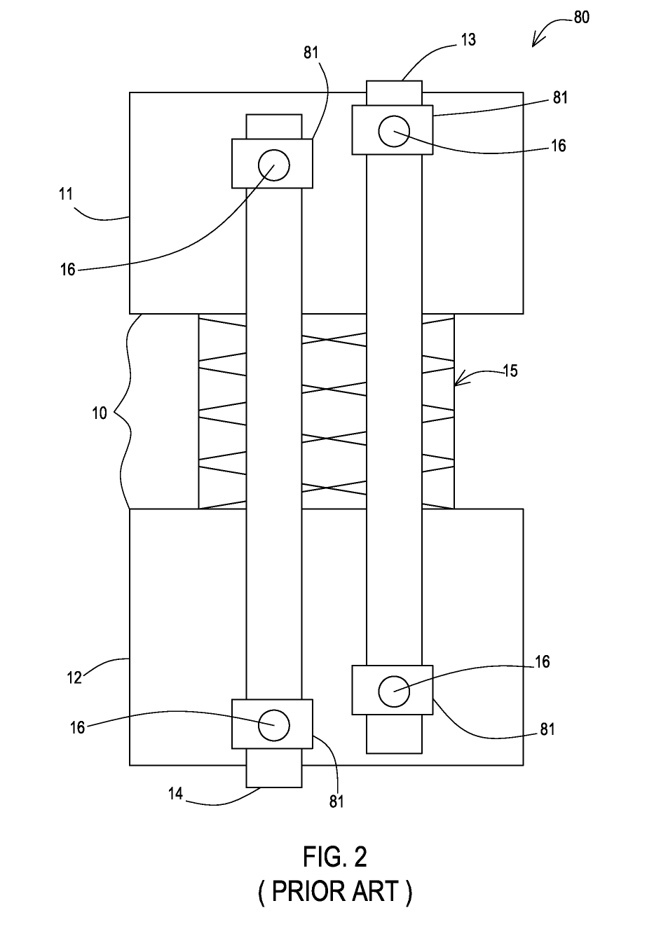

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to devices for connecting portions of one or more vertebral bodies to one another. The present invention is further directed to kits containing the herein described devices in combination with one or more additional kit components such as a tool for engaging with and turning a nut positioned along a counterscrew wormgear of the device. The present invention is even further directed to methods of making and using the herein described devices and kits to connect portions of one or more vertebral bodies to one another.

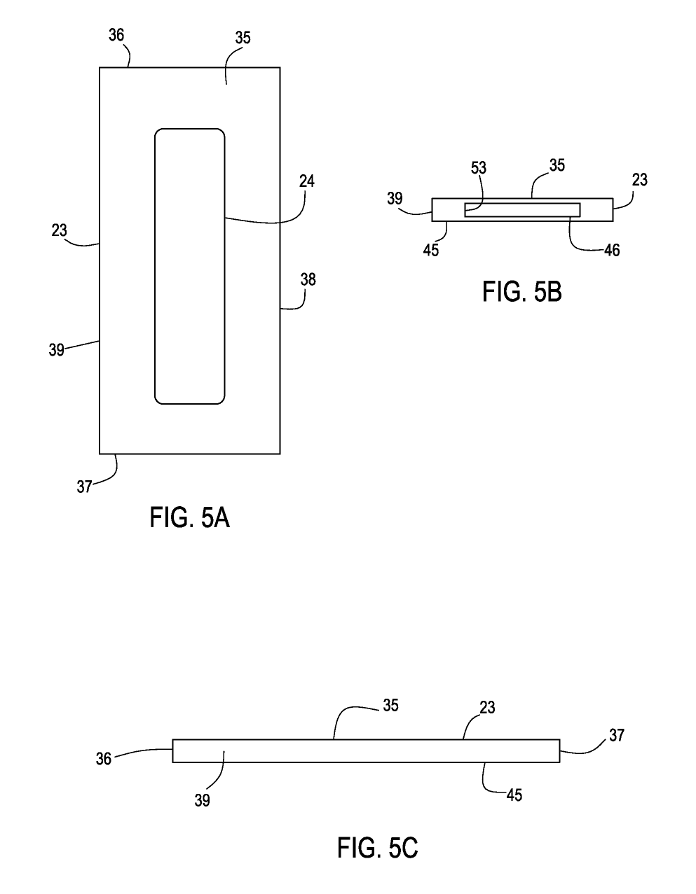

As described herein, the devices and kits of the present invention may comprise, consist essentially of, or consist of one or more of the specifically described components of a given device or kit. Further, the devices and kits of the present invention, as illustratively disclosed herein, may be practiced in the absence of any element, which is not specifically disclosed or described herein. For example, exemplary outer surface of exemplary housing member 23 is shown in the figures, e.g., side surface 39 shown in FIG. 5C, as comprising a relatively flat or smooth surface with no projections thereon. Consequently, although an express negative limitation specifically excluding a projection along side surface 39 is not provided in the present specification, FIG. 5C clearly shows that the present invention encompassed a device without such a projection. In other words, although all possible negative limitations are not specifically recited herein, the figures clearly show that the present invention may encompass devices without any additional device features other than those shown in the figures.

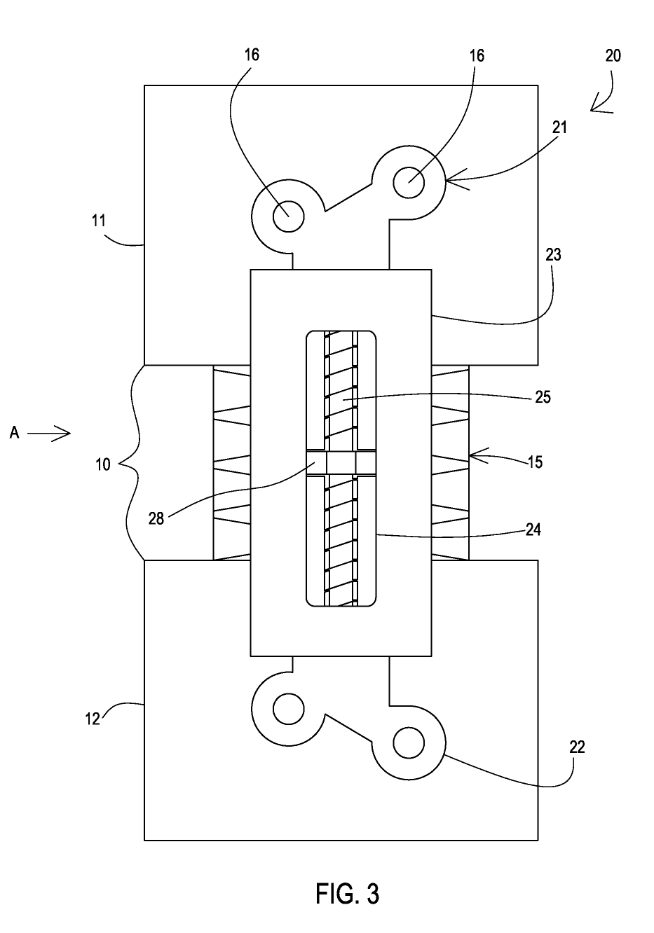

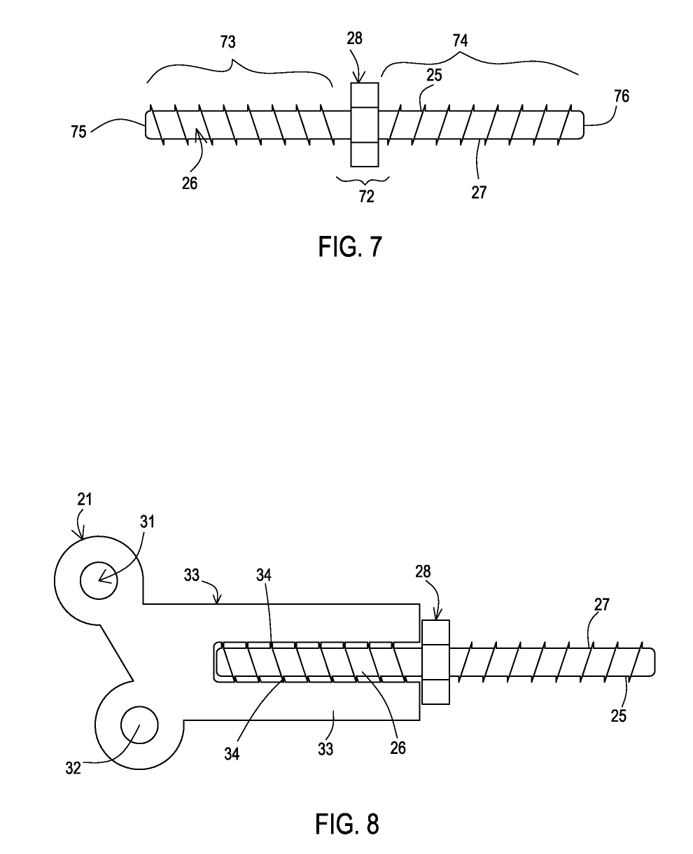

FIG. 3 depicts a frontal view of an exemplary expandable rod device 20 of the present invention. As shown in FIG. 3, exemplary expandable rod device 20 comprises a first member 21 (also referred to herein as first engaging member 21) operatively adapted and sized to connect to a first portion of a vertebral body 11; a second member 22 (also referred to herein as second engaging member 22) operatively adapted and sized to connect to a second portion of a vertebral body 12; and a counterscrew wormgear 25 comprising (i) a nut 28 positioned along a central portion 72 of counterscrew wormgear 25 (see also, for example, FIG. 7), (ii) a set of right-handed threads 26 extending along a first length 73 of counterscrew wormgear 25 between nut 28 and a first end 75 of counterscrew wormgear 25, and (iii) a set of left-handed threads 27 extending along a second length 74 of counterscrew wormgear 25 between nut 28 and a second end 76 of counterscrew wormgear 25 opposite first end 75 of counterscrew wormgear 25. As shown in FIG. 3, counterscrew wormgear 25 is engaged with first and second members 21 and 22 so as to cause first and second members 21 and 22 to move toward or away from one another depending on a direction of rotation of counterscrew wormgear 25 via nut 28.

As shown in FIG. 3, exemplary expandable rod device 20 further comprises exemplary housing member 23. Exemplary housing member 23 is operatively adapted and sized to house (i) at least a portion of first member 21, (ii) at least a portion of second member 22, and (iii) at least a portion of counterscrew wormgear 25. Exemplary housing member 23 further comprises at least one opening 24 therein so as to allow access to nut 28. Desirably, housing member 23 comprises a tubular housing member as shown in FIG. 3 and as further described below.

Exemplary expandable rod device 20 is shown in FIG. 3 attached to first portion of a vertebral body 11 and second portion of a vertebral body 12 via multiple fasteners 16. Each of multiple fasteners 16 is operatively adapted and sized to connect (i) first member 21 to first portion of vertebral body 11, and (ii) second member 22 to second portion of vertebral body 12. Each of multiple fasteners 16 may comprise, for example, a screw.

As further shown in FIG. 3, an exemplary implant 15 may be provided between first portion of vertebral body 11 and second portion of vertebral body 12. As discussed above, although exemplary first portion of vertebral body 11, second portion of vertebral body 12, corpectomy site 10, and exemplary implant 15 are shown as having substantially square shapes, it should be understood that each of exemplary first portion of vertebral body 11, second portion of vertebral body 12, corpectomy site 10, and exemplary implant 15 may independently have any shape necessary for a given surgical procedure.

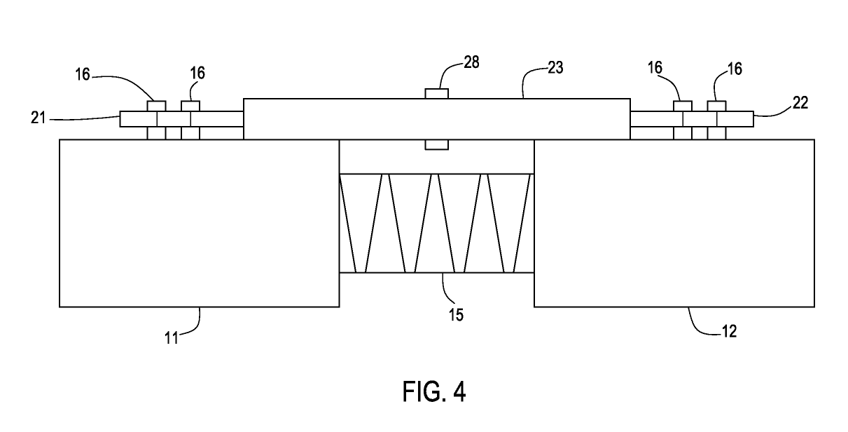

FIG. 4 provides a side view of exemplary expandable rod device 20 shown in FIG. 3 as viewed along direction A. As shown in FIG. 4, multiple fasteners 16 are utilized to attach (i) exemplary first member 21 to first portion of vertebral body 11, and (ii) exemplary second member 22 to second portion of vertebral body 12. It should be noted that although portions of nut 28 are shown extending beyond outer upper and lower surfaces of exemplary housing member 23, exemplary housing member 23 may be provided with outer dimensions so as to completely encompass nut 28.

FIGS. 5A-5C depict frontal, side and ends views of exemplary housing member 23 of exemplary expandable rod device 20. As shown in FIGS. 5A-5C, exemplary housing member 23 comprises a tubular member having a first housing end 36, a second housing end 37 opposite first housing end 36, one or more housing walls (e.g., housing walls having outer upper surface 35, outer lower surface 45, outer side surface 38 and outer side surface 39) extending between first and second housing ends 36 and 37, and a channel 46 extending through housing member 23 from first housing end 36 to second housing end 37. Desirably, exemplary housing member 23 and channel 46 extending therethrough are each sized so that (i) a portion of first member 21, (ii) a portion of second member 22, and (iii) counterscrew wormgear 25 may be positioned within channel 46 so as to be surrounded by channel wall surface 53.

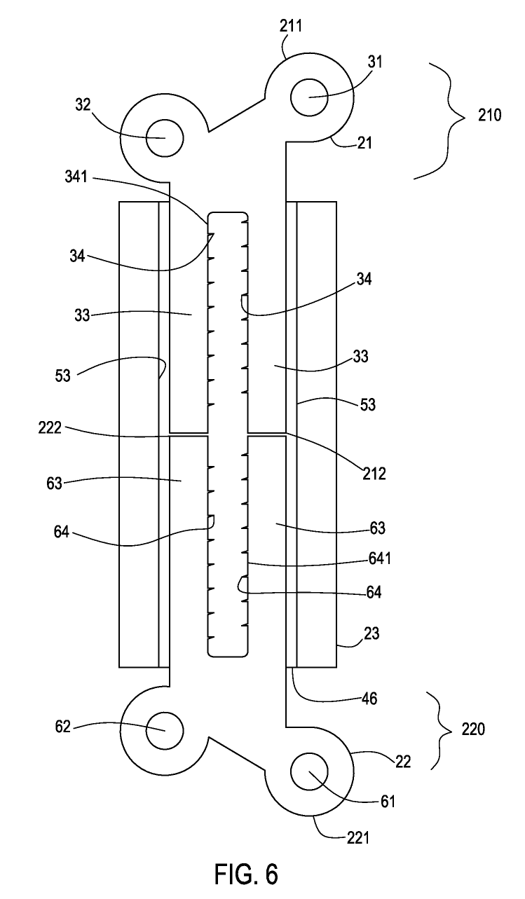

FIG. 6 depicts a portion of exemplary first member 21 and a portion of second member 22 positioned within channel 46 as viewed with upper surface 35 (and the upper wall comprising upper surface 35 and opening 24) of exemplary housing member 23 removed. As shown in FIG. 6, exemplary first member 21 comprises a first engaging member 21 comprising (i) a first engaging member body 210, (ii) a first set of fastening members 31 and 32 positioned proximate a first end 211 of first engaging member 21 and thru first engaging member body 210, (iii) one or more first arm members 33 extending from first engaging member body 210 and toward a second end 212 of first engaging member 21, and (iv) at least one first set of teeth 34 positioned along a surface 341 of one or more first arm members 33. As further shown in FIG. 6, second member 22 comprises a second engaging member 22 comprising (i) a second engaging member body 220, (ii) a second set of fastening members 61 and 62 positioned proximate a first end 221 of second engaging member 22 and thru second engaging member body 220, (iii) one or more second arm members 63 extending from second engaging member body 220 and toward a second end 222 of second engaging member 22, and (iv) at least one second set of teeth 64 positioned along a surface 641 of one or more second arm members 64.

Although not shown in FIG. 6, first set of teeth 34 of first engaging member 21 are engageable with set of right-handed threads 26 of counterscrew wormgear 25, and second set of teeth 64 are engageable with set of left-handed threads 27 of counterscrew wormgear 25. In FIG. 6, exemplary first engaging member 21 and exemplary second set of teeth 64 are shown in an abutting configuration. In other words, as shown in FIG. 6, exemplary first engaging member 21 and exemplary second set of teeth 64 would only separate from one another when engaged and moved by counterscrew wormgear 25.

It should be noted that although each of exemplary first engaging member 21 and exemplary second engaging member 22 are shown as comprising (1) two arm members (i.e., first arm members 33 and second arm members 63), and (2) two sets of teeth (i.e., first sets of teeth 34 second sets of teeth 64), each of exemplary first engaging member 21 and exemplary second engaging member 22 may independently comprise (1) one or more arm members (i.e., first arm members 33 and second arm members 63), and (2) one or more sets of teeth (i.e., first sets of teeth 34 second sets of teeth 64).

FIG. 7 depicts exemplary counterscrew wormgear 25 and exemplary nut 28 suitable for use in exemplary expandable rod device 20 shown in FIG. 3. FIG. 8 depicts exemplary counterscrew wormgear 25 positioned relative to and engaged with exemplary first engaging member 21. In FIG. 8, exemplary first engaging member 21 is shown in an abutting configuration with counterscrew wormgear 25. In other words, as shown in FIG. 8, counterscrew wormgear 25 would only separate from exemplary first engaging member 21 when engaged and moved by counterscrew wormgear 25.

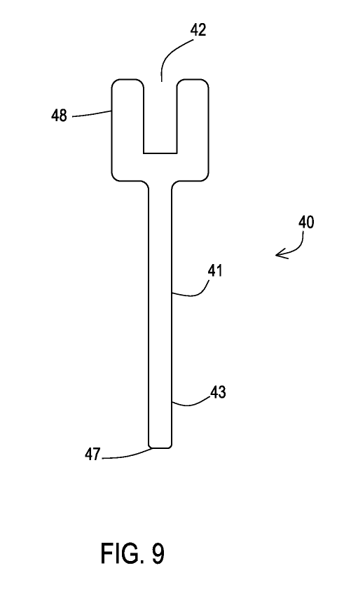

FIG. 9 depicts exemplary tool 40 suitable for engaging with exemplary nut 28 and causing movement of exemplary counterscrew wormgear 25 within exemplary expandable rod device 20 shown in FIGS. 3-4. Exemplary tool 40 comprises a handle 43 at a first end 47, a nut engaging member 42 at a second end 48 opposite first end 47, and a central portion 41 connecting handle 43 with nut engaging member 42. To use, nut engaging member 42 is positioned onto nut 28 and turned in a radial direction relative to counterscrew wormgear 25.

The present invention further comprises any one of the herein described devices in combination with first and second portions of one or more vertebral bodies. For example, the present invention encompasses the exemplary combination of exemplary expandable rod device 20 attached to first portion of a vertebral body 11 and second portion of a vertebral body 12 via first and second members 21 and 22 respectively is shown in FIGS. 3-4.

The present invention is further directed to kits for connecting portions of one or more vertebral bodies to one another. The kits of the present invention may comprise any of the devices described herein (i.e., devices comprising, consisting essentially of, or consisting of one or more of the following components: a first member or first engaging member, a second member or second engaging member, a counterscrew wormgear, and a housing member) in combination with one or more additional kit components. Suitable additional kit components include, but not limited to, multiple fasteners operatively adapted and sized to connect a first and second members or engaging members to first and second portion of one or more vertebral bodies (e.g., multiple screws); and one or more implants for positioning between first and second portions of one or more vertebral bodies.

It should be noted that there are no limitations on the materials used to form each of the device components. In one desired embodiment, each of the above-described device components (i.e., first member or first engaging member 21, second member or second engaging member 22, counterscrew wormgear 25, and housing member 23) comprise, consist essentially of, or consist of radiolucent materials to allow post-operative imaging like MRI, etc.

Further, it should be noted that there are no limitations on the dimensions of each of the device components. In one desired embodiment, the above-described device components (i.e., first member or first engaging member 21, second member or second engaging member 22, counterscrew wormgear 25, and housing member 23) have the following dimensions as shown in Table 1.

TABLE 1
Exemplary Dimensions

| | | | More | Even More |
|---|---|---|---|---|
| Device | | Desired | Desired | Desired |
| Component | Dimension | Range | Range | Range |
| first | overall length | from about | from about | from about |
| member | (from first end 211 to | 10 to about | 20 to about | 30 to about |
| or first | second end 212) | 150 mm | 120 mm | 100 mm |
| engaging | overall width | from about | from about | from about |
| member 21 | (greatest dimension | 5 to about | 10 to about | 15 to about |
| | perpendicular to length) | 40 mm | 30 mm | 20 mm |
| second | overall length | from about | from about | from about |
| member or | (from first end 221 to | 10 to about | 20 to about | 30 to about |
| second | second end 222) | 150 mm | 120 mm | 100 mm |
| engaging | overall width | from about | from about | from about |
| member 22 | (greatest dimension | 5 to about | 10 to about | 15 to about |
| | perpendicular to length) | 40 mm | 30 mm | 20 mm |
| counter- | overall length | from about | from about | from about |
| screw | (from first end 75 to | 10 to about | 20 to about | 30 to about |
| wormgear | second end 76) | 150 mm | 120 mm | 100 mm |
| 25 | overall width | from about | from about | from about |
| | (greatest dimension | 10 to about | 8 to about | 5 to about |
| | perpendicular to length) | 15 mm | 12 mm | 10 mm |
| housing | overall length | from about | from about | from about |
| member 23 | (from first end 36 to | 10 to about | 20 to about | 30 to about |
| | second end 37) | 150 mm | 120 mm | 100 mm |
| | overall width | from about | from about | from about |
| | (from outer side | 5 to about | 10 to about | 15 to about |
| | surface 38 to outer | 40 mm | 30 mm | 20 mm |
| | side surface 39) | | | |
| | overall height/thickness | from about | from about | from about |
| | (from outer side | 10 to about | 8 to about | 5 to about |
| | surface 35 to outer | 15 mm | 12 mm | 10 mm |
| | side surface 45) | | | |

The present invention is further directed to methods of making the disclosed devices and kits for connecting portions of one or more vertebral bodies to one another. In one exemplary embodiment, the method of making a device for connecting portions of one or more vertebral bodies to one another comprises combining (1) first member 21 operatively adapted and sized to connect to first portion of vertebral body 11, (2) second member 22 operatively adapted and sized to connect to second portion of the vertebral body 12, and (3) counterscrew wormgear 25, wherein counterscrew wormgear 25 is engageable with first and second members 21 and 22 so as to cause first and second members 21 and 22 to move toward or away from one another depending on a direction of rotation of counterscrew wormgear 25 via nut 25.

The disclosed methods of making a device or kit for connecting portions of one or more vertebral bodies to one another may further comprise one or more additional steps. Suitable steps include, but are not limited to, engaging counterscrew wormgear 25 with first and second members 21 and 22; positioning the engaged counterscrew wormgear 25 and first and second members 21 and 22 within housing member 25; and providing multiple fasteners 16 (e.g., screws) operatively adapted and sized to connect first and second members 21 and 22 to first and second portions 11 and 12 of the one or more vertebral bodies.

The present invention is even further directed to methods of using the disclosed devices and kits for connecting portions 11 and 12 of one or more vertebral bodies to one another. In one exemplary embodiment, the method of using a device for connecting portions 11 and 12 of one or more vertebral bodies to one another comprises a method of adjusting a position of first portion 11 of one or more vertebral bodies relative to a second portion 12 of the one or more vertebral bodies. The method may comprise (I) attaching first and second members 21 and 22 of exemplary device 20 to first and second portions 11 and 12 of one or more vertebral bodies respectively; and (II) turning nut 28 of counterscrew wormgear 25.

The present invention is described above and further illustrated in the accompanying figures, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A device for connecting portions of one or more vertebral bodies to one another, said device comprising:
a first member operatively adapted and sized to connect to a first portion of one or more vertebral bodies;
a second member operatively adapted and sized to connect to a second portion of the one or more vertebral bodies; and
a counterscrew wormgear comprising (i) a nut positioned along a central portion of said counterscrew wormgear, (ii) a set of right-handed threads extending along a first length of said counterscrew wormgear between said nut and a first end of said counterscrew wormgear, and (iii) a set of left-handed threads extending along a second length of said counterscrew wormgear between said nut and a second end of said counterscrew wormgear opposite said first end of said counterscrew wormgear; said counterscrew wormgear being engaged with said first and second members so as to cause said first and second members to move toward or away from one another depending on a direction of rotation of said counterscrew wormgear via said nut.

2. The device of claim 1, further comprising first and second portions of the one or more vertebral bodies, said first and second members being connected to said first and second portions of the one or more vertebral bodies respectively.

3. The device of claim 1, wherein said first member comprises a first engaging member comprising (i) a first engaging member body, (ii) a first set of fastening members positioned proximate a first end of said first engaging member and thru said first engaging member body, (iii) one or more first arm members extending from said first engaging member body and toward a second end of said first engaging member, and (iv) at least one first set of teeth positioned along a surface of said one or more first arm members, said at least one first set of teeth being engaged with said set of right-handed threads of said counterscrew wormgear; and said second member comprises a second engaging member comprising (i) a second engaging member body, (ii) a second set of fastening members positioned proximate a first end of said second engaging member and thru said second engaging member body, (iii) one or more second arm members extending from said second engaging member body and toward a second end of said second engaging member, and (iv) at least one second set of teeth positioned along a surface of said one or more second arm members, said at least one second set of teeth being engaged with said set of left-handed threads of said counterscrew wormgear.

4. The device of claim 3, further comprising a housing member, said housing member being operatively adapted and sized to house (i) at least a portion of said first member, (ii) at least a portion of said second member, and (iii) said counterscrew wormgear, wherein said housing comprising at least one opening therein so as to allow access to said nut.

5. The device of claim 4, wherein said housing member comprises a tubular member having a first housing end, a second housing end opposite said first housing end, one or more housing walls extending between said first and second housing ends, and a channel extending through said housing member from said first housing end to said second housing end; said (i) portion of said first member, (ii) said portion of said second member, and (iii) said counterscrew wormgear being positioned within said channel.

6. The device of claim 5, further comprising multiple fasteners, said multiple fasteners being operatively adapted and sized to connect (i) said first member to the first portion of the one or more vertebral bodies, and (ii) said second member to the second portion of the one or more vertebral bodies.

7. The device of claim 6, wherein said multiple fasteners comprise multiple screws.

8. The device of claim 7, further comprising first and second portions of the one or more vertebral bodies, said first and second members being connected to said first and second portions of the one or more vertebral bodies respectively via said multiple screws.

9. A kit comprising the device of claim 7 in combination with a tool for engaging with and turning the nut of the counterscrew wormgear.

10. The kit of claim 9, further comprising one or more implants for positioning between first and second portions of one or more vertebral bodies.

11. A device for connecting portions of one or more vertebral bodies to one another, said device comprising:
a first engaging member comprising (i) a first engaging member body, (ii) a first set of fastening members positioned proximate a first end of said first engaging member and thru said first engaging member body, (iii) one or more first arm members extending from said first engaging member body and toward a second end of said first engaging member, and (iv) at least one first set of teeth positioned along a surface of said one or more first arm members;
a second engaging member comprising (i) a second engaging member body, (ii) a second set of fastening members positioned proximate a first end of said second engaging member and thru said second engaging member body, (iii) one or more second arm members extending from said second engaging member body and toward a second end of said second engaging member, and (iv) at least one second set of teeth positioned along a surface of said one or more second arm members;
a counterscrew wormgear comprising (i) a nut positioned along a central portion of said counterscrew wormgear, (ii) a set of right-handed threads extending along a first length of said counterscrew wormgear between said nut and a first end of said counterscrew wormgear, and (iii) a set of left-handed threads extending along a second length of said counterscrew wormgear between said nut and a second end of said counterscrew wormgear opposite said first end of said counterscrew wormgear, said counterscrew wormgear being engaged with said first and second engaging members so as to cause said first and second engaging members to move toward or away from one another depending on a direction of rotation of said counterscrew wormgear via said nut; and
a tubular housing member comprising a first housing end, a second housing end opposite said first housing end, one or more housing walls extending between said first and second housing ends, a channel extending through said tubular housing member from said first housing end to said second housing end, and at least one opening therein so as to allow access to said nut; wherein (i) a portion of said first engaging member, (ii) a portion of said second engaging member, and (iii) said counterscrew wormgear are positioned within said channel.

12. The device of claim 11, further comprising multiple screws, said multiple screws being operatively adapted and sized to connect (i) said first engaging member to a first portion of the one or more vertebral bodies, and (ii) said second engaging member to a second portion of the one or more vertebral bodies.

13. The device of claim 12, further comprising first and second portions of one or more vertebral bodies, said first and engaging second members being connected to said first and second portions of the one or more vertebral bodies respectively via said multiple screws.

14. A kit comprising the device of claim 12 in combination with a tool for engaging with and turning the nut of the counterscrew wormgear.

15. The kit of claim 14, further comprising one or more implants for positioning between first and second portions of one or more vertebral bodies.

16. A kit for connecting portions of one or more vertebral bodies to one another, said kit comprising:
a device comprising:
a first member operatively adapted and sized to connect to a first portion of one or more vertebral bodies;
a second member operatively adapted and sized to connect to a second portion of the one or more vertebral bodies; and
a counterscrew wormgear comprising (i) a nut positioned along a central portion of said counterscrew wormgear, (ii) a set of right-handed threads extending along a first length of said counterscrew wormgear between said nut and a first end of said counterscrew wormgear, and (iii) a set of left-handed threads extending along a second length of said counterscrew wormgear between said nut and a second end of said counterscrew wormgear opposite said first end of said counterscrew wormgear; said counterscrew wormgear being engageable with said first and second members so as to cause said first and second members to move toward or away from one another depending on a direction of rotation of said counterscrew wormgear via said nut; and
a tool for engaging with and turning the nut of the counterscrew wormgear.

17. The kit of claim 16, wherein said device further comprises a tubular housing member comprising a first housing end, a second housing end opposite said first housing end, one or more housing walls extending between said first and second housing ends, a channel extending through said tubular housing member from said first housing end to said second housing end, and at least one opening therein so as to allow access to said nut when said counterscrew wormgear is positioned within said channel; wherein said channel is sized so as to enable (i) a portion of said first member, (ii) a portion of said second member, and (iii) said counterscrew wormgear to be positioned therein.

18. The kit of claim 17, further comprising one or more implants for positioning between first and second portions of one or more vertebral bodies.

19. A method of adjusting a position of a first portion of one or more vertebral bodies relative to a second portion of the one or more vertebral bodies, said method comprising:
attaching the first and second members of the device of claim 1 to the first and second portions of the one or more vertebral bodies respectively; and
turning the nut of the counterscrew wormgear.

20. A method of adjusting a position of a first portion of one or more vertebral bodies relative to a second portion of the one or more vertebral bodies, said method comprising:
attaching the first and second members of the device from the kit of claim 18 to the first and second portions of the one or more vertebral bodies respectively;
positioning one or more implants between the first and second portions of the one or more vertebral bodies; and
turning the nut of the counterscrew wormgear with the tool.
